(19) United States
(12) Patent Application Publication
(10) Pub. No.: US 2009/0155245 A1
(43) Pub. Date: Jun. 18, 2009

(54) Treatment for Organ Regeneration with Combination of Drug and Biologics

(76) Inventors: Carrie H. Fang, Pittstown, NJ (US); Sridevi Dhanaraj, Raritan, NJ (US); Jeffrey C. Geesin, Doylestown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/330,704

(22) Filed: Dec. 9, 2008

Related U.S. Application Data
(60) Provisional application No. 61/014,538, filed on Dec. 18, 2007.

(51) Int. Cl. A61K 35/12; A61K 35/28; A61K 39/395; A61P 13/12

(52) U.S. Cl. 424/130.1; 424/93.7

(57) ABSTRACT

Combination therapies of drugs and biologics for treatment of acute and chronic organ degeneration are disclosed. More specifically, the use of morphogens, stem cells, and anti-inflammatories or inhibitors for the renin-angiotensin-aldosterone system (RAAS) for the treatment of acute and chronic renal disease is disclosed.

### FIELD OF THE INVENTION

The invention relates to combination therapy of drugs and biologics for treatment of acute and chronic organ degeneration. More specifically, the invention relates to use of morphogens, stem cells, and anti-inflammatories or inhibitors for the renin-angiotensin-aldosterone system (RAAS) for the treatment of acute and chronic renal diseases.

### BACKGROUND OF THE INVENTION

The mammalian renal system serves primary roles both in the removal of catabolic waste products from the bloodstream and in the maintenance of fluid and electrolyte balances in the body. Renal failure is, therefore, a life-threatening condition in which the build-up of catabolites and other toxins, and/or the development of significant imbalances in electrolytes or fluids, may lead to the failure of other major organ systems and death. Renal failure is classified as “acute” or “chronic”, which is a result of inadequate intrinsic regenerative/repair mechanisms. The mortalities in both conditions are high. Therefore, there is considerable drive to develop improved therapies for renal failure with the capacity to replace a wider range of the kidney's functions, thereby reducing morbidity, mortality and the overall economic impact associated with these conditions.

A number of proteins have now been identified which appear to act as morphogenetic or growth factors, regulating cell proliferation and/or differentiation. One group of morphogenetic proteins, referred to herein as “morphogens,” includes members of the family of osteogenic proteins/bone morphogenetic proteins (OP/BMPs), which were initially identified by their ability to induce ectopic endochondral bone morphogenesis. BMPs are low molecular weight noncollagenous glycoproteins that belong to an expanding TGF-β superfamily. BMPs are expressed in a variety of tissues during development and in adult tissues, implying a myriad of functions of this family of proteins. They cause mesenchymal cells to differentiate into chondrocytes, which create a cartilage matrix that mineralizes and then is replaced by bone (endochondral ossification), or they may cause mesenchymal cells to directly differentiate into osteoblasts that produce matrix and regulate callus and new bone formation (intramembranous bone formation). Therefore, BMPs are often used in regeneration of bone and soft tissues including cartilage, ligament or tendon. Recently, evidence for the involvement of BMPs in metanephric development emerged when renal phenotypes were observed in BMP-7/OP-1 null mutant mice. Since then, several other BMPs (BMP-2, -4, -3 and -5), BMP receptor serine/threonine kinases (ActR-IA, BMPR-IA, -IB and BMPRII) and BMP signal transduction molecules (Smads: Smads1, 2, 3, 4, 5 and 6) have been implicated in mammalian metanephric development. It appears that particular BMPs have pivotal roles in specific aspects of metanephric development.

The growth/differentiation factor-5 (GDF-5) belongs to the subfamily of the highly conserved class of BMP signaling molecules, known to play a variety of roles not only in musculoskeletal system, but also have functional significance in central nervous system, cornea as well as other systems. GDF-5 shares many structural and functional similarities with BMP-7. While roles of BMPs 2, 4, and 7 in developing kidney have been characterized in more depth, roles of GDF-5 in kidney are largely unknown. We have demonstrated that rhGDF-5 and its corresponding receptors are abundantly expressed in all layers of normal human kidney tissue (data not shown), implying a potential role of GDF-5 synthesized in kidney as a paracrine regulator of bone growth that is consistent with the role of the kidneys in both calcium regulation and bone homeostasis.

An important emerging paradigm in the understanding of renal disease is the recognition of the central role of inflammation in the initiation and progression of acute and chronic kidney injury. These advances have led to an increasing awareness of the importance in control or inhibition of these inflammatory disorders. There is a growing body of evidence indicating that p38 mitogen-activated protein kinase (MAPK) is a key mediator in organ dysfunction relating to the inflammatory state, and acts as an important mediator in the intracellular signal pathway for proliferation, differentiation, and production of proinflammatory cytokines such as TNF-alpha, IL-1beta, TGF-beta1 and MCP-1/CCL-2 (monocyte chemoattractant peptide-1 or CC chemokine ligand-2), the later two of which are critical mediators in promoting excessive inflammation and extracellular matrix deposition in kidney fibrosis.

Hemodynamic stability in humans is highly dependent on proper functioning of the rennin-angiotensin-aldosterone system (RAAS). This cascade plays an integral role in maintaining normal hemodynamics and electrolyte balance. Unregulated and excessive production of angiotensin II is associated with renal injury that can become progressive and irreversible. Many basic studies and clinical studies in humans have shown that inhibition of the RAAS reduces the injurious effects of angiotensin II in diabetic and nondiabetic nephropathies.

Alternatively, the RAAS can also be activated by a low NaCl concentration in the macula densa or by sympathetic activation.

Ace inhibitors target the ace gene product, angiotensin converting enzyme. ARBs target receptors for angiotensin and aldosterone receptor antagonists interfere with the interaction between the ACE upregulated aldosterone and its specific receptors, resulting in downstream reduction of angiotensin, decreased aldosterone secretion and attenuation of the signal transduction through aldosterone receptor. The decrease in aldosterone decreases sodium and water resorption in the kidney and decreases potassium excretion. Through their action on the bradykinin pathway they also increase production of nitric oxide and induce vasodilation.

While the kidney has an intrinsic ability to regenerate tubular epithelium after injury, its capacity is limited to an early stage of acute conditions. In kidney failure patients, especially those with chronic kidney diseases, loss of kidney function is associated with significant tissue damage and exhaustion of intrinsic source of regeneration. Supplement of progenitor cells to the site of injury to repopulate and reconstitute the damaged tissue would be essential or even necessary to restore the lost function. Numerous reports have shown that BM can be a source of cells contributing to regeneration of different renal cells especially after renal injury, including tubular epithelial, mesangial and endothelial cells. Cell-based therapies with BM or other progenitor cells may thus have the advantage of acting through multiple mechanisms in disorders with highly complex pathophysiology such as renal failure.

Kidney diseases are caused by a complicated disordered interaction of multiple genes and biological pathways. Correction of these biological disorders needs to be addressed through multiple agents with different mechanism of actions. Therefore, the multifactorial nature of the kidney diseases may require a combination of therapy including a growth factor, progenitor cells with drugs to correct hemodynamic disorders or inflammation.

### SUMMARY OF THE INVENTION

The present invention is directed to methods of treatment for patients at risk of acute or chronic renal failure by administering a combination of certain morphogens, cells and anti-inflammatories or inhibitors for the RAAS in any order, or simultaneously as a mixture of the three. The morphogens include the morphogen itself, morphogen derivatives that maintain original function but with improved solubility, inducers of those morphogens, or agonists of corresponding morphogen receptors. In a preferred embodiment, the morphogen is rhGDF-5 delivered intravenously, locally through intraperitoneal, intrarenal artery, or renal subcapsular injection, or locally by implantation in matrices. Cells for administration can include therapeutically beneficial cells, including autologous or allogenic mesenchymal stem cells (MSCs), adult stem cells from any tissue, stem cells from any developmental stage and renal cells induced with morphogens. A preferred embodiment of the cells is bone marrow mesenchymal cells. Anti-inflammatories can be from a class of compounds called P38 MAP Kinase inhibitors, antibodies against pro-inflammatory cytokines, antibodies against chemoattractant molecules or cell cycle inhibitors. Inhibitors for RAAS can be any classes of compounds that attenuate RAAS pathways. A preferred embodiment of this class is ACE inhibitor.

### BRIEF DESCRIPTION OF THE FIGURES

FIG. 1. Shows the weighted ranking for the efficacy of different treatments on various parameters in 5/6 Nx study.

Both sham and 5/6 Nx performed rats were treated 4 weeks post surgery with different reagents as indicated. The effects of the treatment were analyzed every two weeks through blood and urine sample analysis consisting of hematology, serum and urine chemistry and proteinuria. After 10 weeks of treatment, kidney samples were taken from the right kidney and evaluated using histopathology for glomerulorsclerosis, interstitial fibrosis and tubular degemation. All the data were converted to a weighted ranking system from 1 to 10 and averaged for the 10 weeks. A better efficacy from a treatment receives a higher score and the maximum score is the level for sham group. V: Vehicle; M: rhGDF-5; E: Enalapril; C: MSC; SC:serum creatinine concentration; BUN: blood urea nitrogen; PU:UC: ratio of proteinuria to urine creatinine; GS: glomerulosclerosis; IF+TD interstitial fibrosis and tubular degeneration.

FIG. 2A. Shows the effect of the treatment groups on the renal injury score.

FIG. 2B. Shows the effect of the treatment groups on UUO induced renal injury (A) and fibrotic gene expression in mice.

FIG. 3A. Shows the efficacy of rhGDF-5 and combination of rhGDF-5 and Enalapril on renal protection in diabetic rats. Diabetic rats treated with combination of rhGDF-5 and Enalapril has the highest survival rate.

FIG. 3B. Shows the efficacy of rhGDF-5 and combination of rhGDF-5 and Enalapril on renal protection in diabetic rats. Combination therapy reduced tubular epithelial cell vaculolation judged by H&E histology staining.

FIG. 3C. Shows the efficacy of rhGDF-5 and combination of rhGDF-5 and Enalapril on renal protection in diabetic rats. Combination of rhGDF-5 and Enalapril reduced BUN level (mg/dL) at early time point (indicated by an downward arrow).

FIG. 3D. Shows the efficacy of rhGDF-5 and combination of rhGDF-5 and Enalapril on renal protection in diabetic rats. rhGDF-5 has comparable efficacy on reducing urine micro albumine level (mg/dL).

### DETAILED DESCRIPTION OF THE INVENTION

In order to move clearly and concisely to point out the subject matter of the claimed invention, the following definitions apply.

The term “acute renal failure” (ARF) means a rapidly progressive loss of renal function, generally characterised by oliguria, body water and body fluids disturbances; and electrolye derangement.

Ther term “chronic renal failure” (CRF) means a slowly progressive loss of renal function over a period of months or years and defined as an abnormally low glomerular filtration rate, which is usually determined indirectly by the creatinine level in blood serum.

The term “mammals at risk” means mammals at risk of renal failure, or with acute or chronic renal failure, or in need of renal replacement therapy such as dialysis.

The term “blood urea nitrogen” (BUN) means the level of urea nitrogen in blood, which comes from urea, a substance secreted by the liver as a waste product of the digestion of protein and removed from the blood by the kidneys. It is a measure of renal function.

The term “creatinine” (CR) means 2-amino-1-methyl-5H-imidazol-4-one, which is a breakdown product of creatine phosphate in muscle, and is usually produced at a fairly constant rate by the body (depending on muscle mass), mainly filtered by the kidney. Measurement of serum creatinine is the most commonly used indicator of renal function.

The term “morphogen” means a substance governing the pattern of tissue development and, in particular, the positions of the various specialized cell types within a tissue. It spreads from a localized source and forms a concentration gradient across a developing tissue.

The term “osteogenic proteins morphogen” (OP morphogen) means a protein orgininally identified by their ability to induce ectopic endochondral bone morphogenesis

The term “bone morphogenetic proteins” (BMPs) means a group of growth factors and cytokines known for their ability to induce the formation of bone and cartilage. In the present inveniton, BMPs are the growth factors of low molecular weight noncollagenous glycoprotein in the TGF-β superfamily.

The term “Transforming growth factor β (TGF-β”) means a multifuntional peptide that controls cell proliferation, cell differentiation and embryonic development.

The term “Growth and Differentiation factor-5 (GDF-5)” means a protein belonging to the TGF-β superfamily that is expressed in both developing and adult musculoskeletal and central nervous system as well as many other systems in the body. “GDF-5” herein, is ment to include all synonyms, variants, and mutations of GDF-5 protein moleclue, including but not limited to GDF-5, mGDF-5, hGDF-5, MP-52, LAP-4, radotermin, CDMP-1,C465A,and rhGDF-5 wherein rhGDF-5 is the exemplary member of the group. It is also understood to include monomeric GDF-5 proteins, which have also been shown to be biologically active.

The term “stem cell” means primal cells that retain the ability to renew themselves through mitotic cell division and can differentiate into a diverse range of specialized cells. Wherein, MSC are an exemplary member.

The term “anti-inflammatories” means compounds or anitbodies against pro-inflammatory cytokines, chemoattractant molecules, or cell cycle inhibitors. The class of P38 MAP kinase inhibitors is an exemplary member of “anti-inflammatories”.

The term “inhibitors for renin-angiotensin-aldosterone system, or RAAS inhibitors” means classes of compounds that down regulate the activity of this system, including direct inhibitor of renin, inhibitors of angiotensin-converting enzyme (ACE), antagonists for angiotensin II type I (AT1) receptor and aldosterone receptor. “Enalapril” means 1-[2-(1-ethoxycarbonyl-3-phenyl-propyl)aminopropanoyl]pyrrolidine-2-carboxylic acid, which is a representative molecule of an angiotension converting enzyme (ACE) inhibitor used in treatment of hypertension and some types of chronic heart failure. Enalapril is in a group of ACE inhibitors known as the dicarboxylate-containing ACE inhibitors. It is marketed by Merck & Co. (Merck, Sharp, & Dohme) under the trade names, RENITEC® and VASOTEC®.

We have investigated the use of a combination therapy for acute and chronic renal failure in mammals. There are many causes that can trigger the pathophysiological mechanism that lead to renal failure. Renal failure is characterized by the condition in which the kidneys fail to function adequately, with a consequence of loss of hemostatic equilibrium. The primary marker of renal failure is a sharp increase in the concentration of the nitrogenous components in the blood and a profound decrease in the glomerular filtration rate.

There are many current treatments for acute and chronic renal failure. The present invention embodies a combination treatment that is meant to slow inflammation or control hemodynamic disorder of the kidneys while using therapeutic stem cells coupled with morphogenic proteins to effectively facilitate structural and functional regeneration. An object of the present invention is to provide treatment of conditions that place any mammal at risk of acute and chronic renal failure.

We undertook several studies that involved the administration of certain drug and biologic compounds systematically to test theraputic properties. In a prefered embodiment the therapeutic agent includes morphogens (see Examples 1 and 2). The protein morphogens in the present invention are members of the BMP family. Specifically, the morphogen utilized is GDF-5. GDF-5 shows a close structual relationship to bone morphogenic proteins and plays a crucial role in skeletal morphogenesis. In particular, rhGDF-5 is reported as a suitable factor for enhancing healing in bone defects and inducing ectopic bone formation. We have observed that both GDF-5 ligand and its specific receptors are shown to be expresesed in all layers of the kidney tissue. This implies an important role of GDF-5 synthesized in the kidneys as a paracrine regulator of bone growth that is consistant with the role of rhGDF-5 in both calcium regulaiton and bone homeostasis.

In another preferred embodiment the therapeutic agent is stem cells (see Example 1). Bone marrow derived MSCs are prefered embodiment of stem cells in this invention because MSCs can be obtained from autologous source. MSCs are multipotent postnatal stem cells that have been used for the treatment of bone defects, and graft versus-host diseases. In the present invention MSCs are utilized for grafting and as source of trophic factors. These cells can be isolated from various tissues including adult bone marrow, fetal tissues and placenta. In the present invention the MSCs were isolated from bone marrow-adherent cells isolated from adult male Lewis rats.

In addition to MSCs an ACE inhibitor, Enalapril, can be added. Enalapril is a potent inhibitor for the RAAS pathway (see Example 1). Enalapril is commonly used to slow the progression of kidney disease due to high blood pressure or diabetes. A recent development in the treatment of the renal disease is the recognition of the role of an activated RAAS in the initiation and progression chomic renal injury. In the present embodiment, Enalapril is used to slow the progression of kidney degeneration while the theraputic agents are used to rebuild kidney tissue.

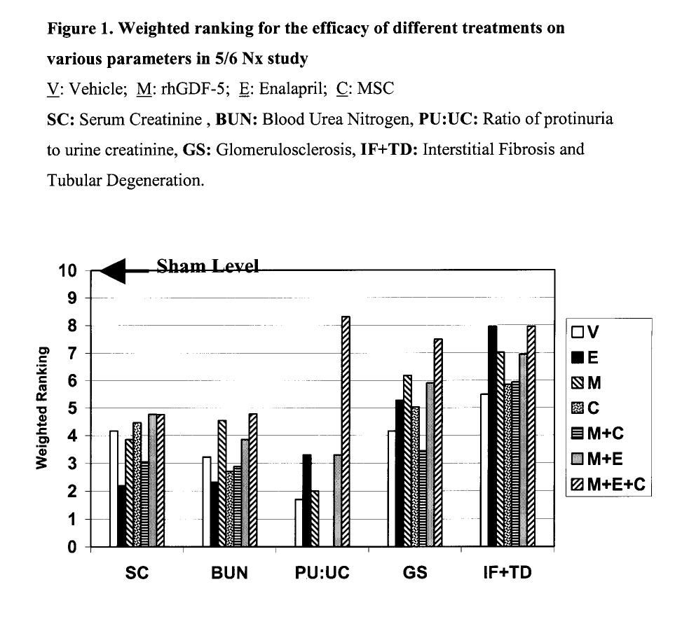

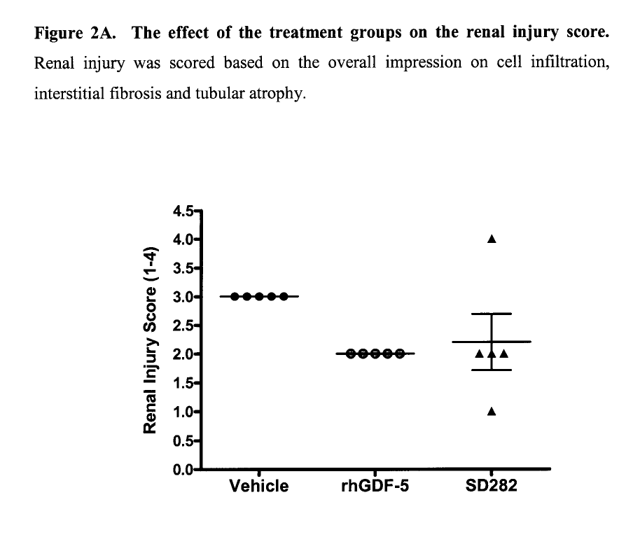

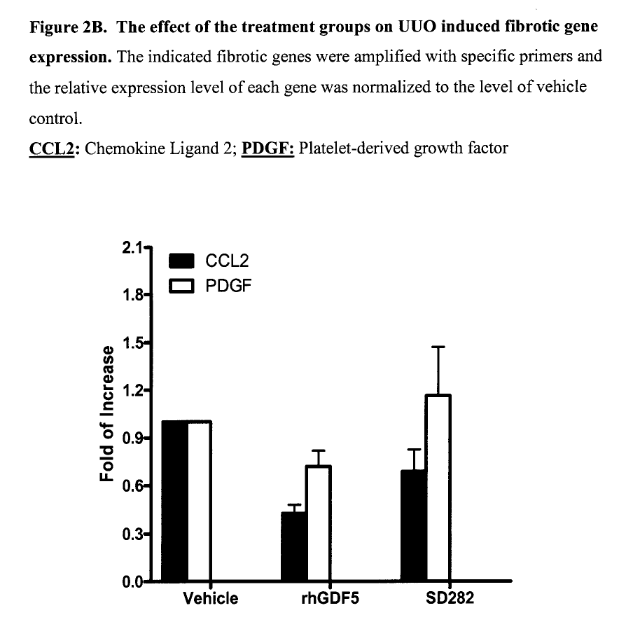

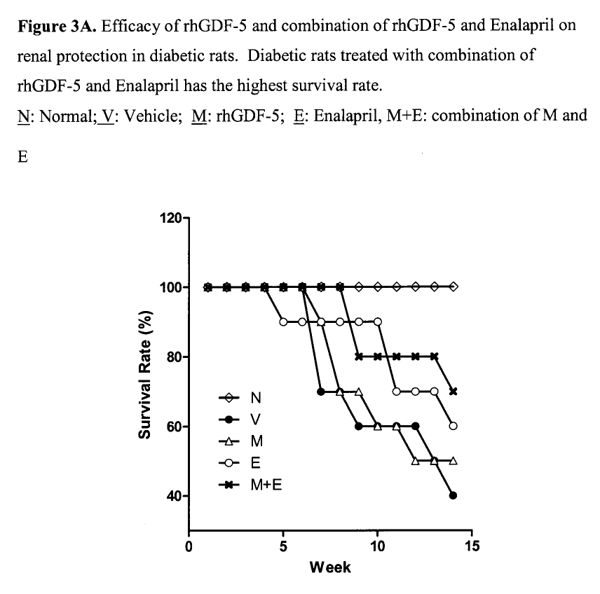

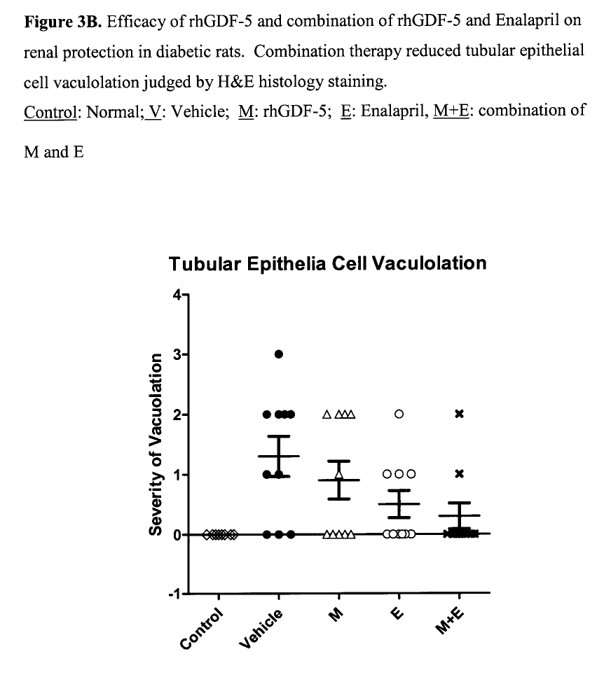

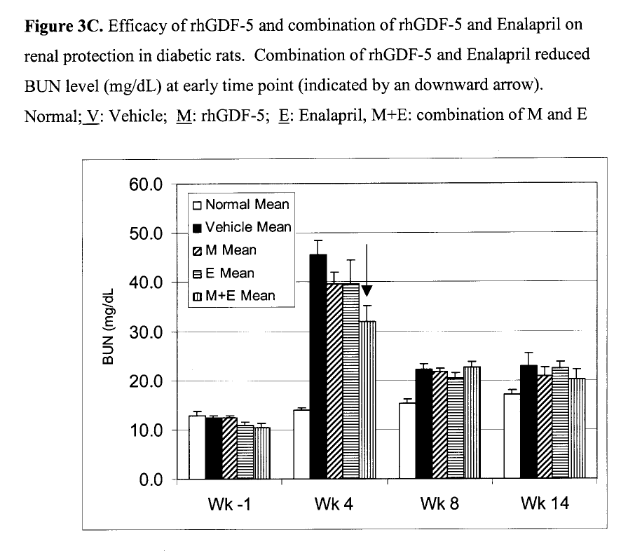

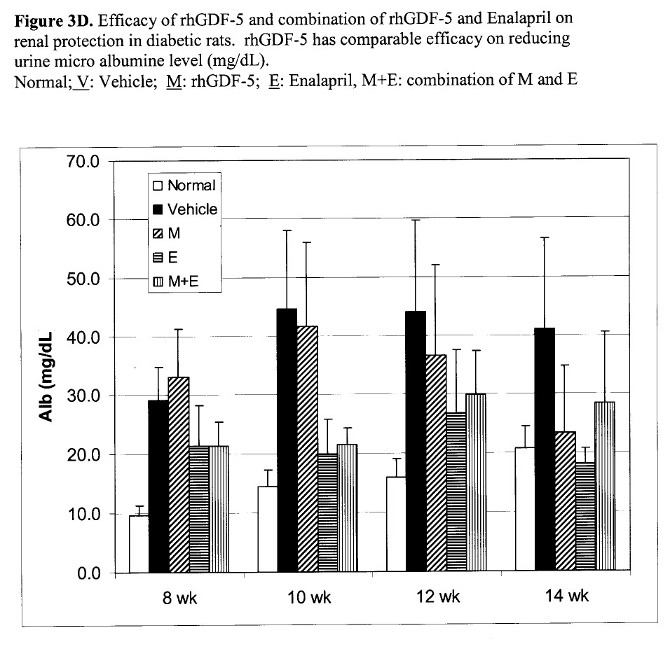

We conducted experiments to show the theraputic adavantages of using morphogens, stem cells and ACE inhibitor in combination to treat chomic renal disease (see Example 1). The data on various paramaters was converted into a weighted ranking system from 1-10. FIG. 1 represents the averages during the course of the 10 wks. A better efficacy from a treatment in reducing serum creatinine, blood urea nitrogen and proteinuria receives a higher ranking and the maximun rank is the sham level.

The results showed postive trends that a triple combination therapy using rhGDF-5, MSC and Enalapril reduced glomerulosclerosis (p<0.1) at 10 wks after treatement as compared to the groups treated with either vehicle or all reagents individually administered or double combination. Additionally combination therapy of all three reagents showed a trend of reduction in proteinurea as compared to the groups treated with either vehicle or each reagent individually adminstered or double combination. Proteinuria, long regarded as merely a marker for glomerular injury, has now been proposed to play an important role in contributing disease progression. The visibly higher ranking for the effect on reduction of proteinuria suggest the potential of the triple combination therapy in slowing or halt the disease progression for chronic kidney diseases.

In another embodiment, an anti-inflammatory, SD282 (1, 2), was included, which belongs to the class of P38 MAP kinase inhibitors (see Example 2). Anti-inflammatory refers to the property of a substance or treatment that reduces inflammation. Anti-inflammatory drugs make up one half of analgesics, remedying pain by reducing inflammation as opposed to opioids which affect the brain.

The theraputic effects of rhGDF-5 was further tested along with SD282 in Unilateral Ureter Obstruction Model (UUO) in the present invention. Mice were subjected to unilateral ureter obstruction and treatment started on the same day as the UUO surgery. After 12 days of treatment, the obstructed kidneys were harvested for Hematoxylin and Eosin (H&E) staining and RT-PCR analysis. As shown in FIG. 2A, compared to the vehicle control in which animals developed severe renal injury, histology scoring showed all animals treated with rhGDF-5 have reduced renal structural damage. Gene expression analysis displayed decreased levels of chemokine ligand 2 (CCL2) and Platelet-derived growth factor (PDGF) gene expression (FIG. 2B) compared to controls. CCL2 induces recruitment of monocytes, T lymphocytes, eosinophils, and basophils and is responsible for many inflammatory reactions to disease, and has been linked to recruitment of osteoclast precursors. PDGF plays a role in embryonic development, cell proliferation, cell migration, and angiogenesis. The decreased expression of CCL2 by rhGDF-5 and SD282 and PDGF by rhGDF-5 in this animal model might be the mechanism of theraputic effects observed in reduction of renal injury by rhGDF-5 and SD282.

The following examples are meant only to be illustrative in nature of the present invention, and not to be limited in scope. One skilled in the art would easily conceive of other embodiments that would be considered within the scope of the present invention.

### EXAMPLE 1

The efficacy of a novel therapy based on treatment groups rhGDF-5, Enalapril, and bone marrow mesenchymal stem cells and combinations thereof in a 5/6 nephrectomy rats was examined.

Inbred Lewis rats were chosen due to involvement of bone marrow cell transplant in the study. Female LEW/H 1a®CVF® (Colony No. 38) weighing 250-300 gm that underwent sham or 5/6 nephrectomy (Nx) procedures were obtained from Hilltop Lab Animals, Inc., Scottdale, Pa. They were housed in a constant temperature room with a 12 hr light and 12 hr dark cycle. Purina Certified Diet and filtered tap water was provided to the animals as desired.

5/6 nephrectomy was achieved by right nephrectomy followed by a surgical resection of upper ⅓ and lower ½ half of left kidney. Four weeks after surgery, when Nephrectomized (Nx) animals had overt blood urea nitrogen and serum creatinine level, they were randomly assigned to groups by a weight-ordered distribution such that individual body weights did not exceed ±20% of mean weight. There were 2 sham groups, vehicle (0.1 N HCl and 1×PBS) and rhGDF-5 and 7 Nx groups: vehicle, rhGDF-5, Enalapril, MSCs, combination rhGDF-5 and Enalapril, combination rhGDF-5 and MSC, and combination rhGDF-5, MSC and Enalapril.

rhGDF-5 was administrated 3 times per week at 50 μg/kg by ip. Enalapril was administered daily at 10 mg/kg by gavage. MSCs were administered once on day 1 through iv at 3×10⁶/kg and combination treatments followed individual route for each drug. Effects of these treatments on kidney function was evaluated every two weeks by monitoring clinical observations, body weights, urine volume and duration of collection, and clinical pathology parameters through blood and urine sample analysis (hematology, serum chemistry, and urine chemistry). Residual urine samples were also evaluated for protein levels, beginning on Day 28, by AniLytics, Inc. On Day 70, surviving animals were euthanized and subjected to a limited necropsy, consisting of collection of the right kidney (2 sham groups) or the remaining third of the right kidney (all the Nx groups). Collected tissues were evaluated histopathologically for glomerulorsclerosis, interstitial filtration, and tubular degeneration.

The effect of the treatment groups on various parameters including both histological and physiological measurements. The data were converted into weighted ranking system from 1-10 and represents the averages during the course of 10 weeks. As shown in FIG. 1, a better efficacy from a treatment in reducing serum creatinine, blood urea nitrogen or proteinuria gives a higher ranking. We look for the effect of a treatment that was not only better than vehicle but also better than the Enalapril, a positive control for the study. The overall observation was that there was no statistically significant difference in the effect of these treatment groups on all parameters from vehicle and positive controls. However, there is a positive trend that the triple combination shows a marginal effect across several parameters and rhGDF-5 alone also has some effect on reducing blood urea nitrogen levels.

### EXAMPLE 2

The efficacy of rhGDF-5 was further tested along with SD282 in a mouse UUO model. Treatment started on the same day of the UUO, and included rhGDF-5 and SD282. At the end of the 12-day treatment, histology scoring and gene expression analysis was preformed on endpoint measurements.

The animals used in this study were handled and maintained in accordance with all applicable sections of the Final Rules of the Animal Welfare Act regulations (9 CFR), the Public Health Service Policy on Humane care and Use of Laboratory Animals, the Guide for the Care and Use of Laboratory Animals. The protocol and amendments or procedures involving the care and use of animals in this study was reviewed and approved by the Testing Facility Institutional Animal Care and Use Committee prior to the initiation of such procedure.

Unilateral Ureter Obstruction Model (“UUO”) was used because it is a commonly used mouse model for progressive tubulointerstitial fibrosis in the kidney. Male C57BL/6 mice were purchased at age 6 to 8 weeks from Jackson Labs (Bar Harbor, Me.). They were housed in an animal facility that was maintained at 25° C. with a 12-hour light-dark cycle. They were fed standard rodent chow containing 22% protein (Purina, St. Louis, Mo.) and provided tap water to drink as desired.

At age 8 to 10 weeks mice were anesthetized with 60 mg/kg pentobarbital administered as an intraperitoneal injection. UUO was created by placing 2 sutures around the right mid ureter. The abdomen was closed in 2 layers, and animals were placed under a warming light and observed until they recovered from the procedure.

The vehicle control for rhGDF-5 consisted of a 0.1 N Hydrochloric Acid (HCl) (J.T. Baker, lot #B07P00) solution. The vehicle control for the Enalapril consisted of a 1× Phosphate Buffered Saline (PBS) solution (Cambrex/BioWhittaker, lot#01111802) of pH 7.3-7.7. A concentrated stock solution of was made by dissolving rhGDF-5 (JJRT, lot #2142121) in 0.1N HCL at 500 μg/ml. Synergistic mesenchymal stem cells were isolated from male Lewis rats (150-160 g). The cells were cultured and selected in MEM-a medium with 10% FCS. Enalapril (lot#055K1446) was purchased from Sigma in powder form. SD282 (1, 2) was prepared daily by dissolving into 0.5% methlycelluslose (Sigma, M7027, lot#064K0038).

Experimental animals were treated with rhGDF-5, 3 times per week, via ip injection at 300 μg/kg and SD282, twice, daily through gavage at 60 mg/kg dissolved in 0.5% methylcellulose. Control mice received an equal volume of 0.5% methylcellulose vehicle. Treatment dose was selected based on a review of the literature and represented the highest dose that was free of side effects in other experimental studies. The duration of treatment was 12 days and started on the same day of UUO surgery. Mice were sacrificed after 12 days of treatment. The obstructed kidney was harvested for RT-PCR (3) analysis as well as histology evaluation on H&E staining (4).

The data indicates that rhGDF-5 alone has a reduced renal injury score (FIG. 2A) and prevents chemokine ligand 2 (CCL2) and platelet-derived growth factor (PDGF) gene expression. (FIG. 2B), the effect of which may be the mechanism for the observed reduction in renal injury score.

### EXAMPLE 3

One could also combine rhGDF-5 with Angiotensin II receptor antagonists also known as angiotensin receptor blockers (ARBs), AT₁-receptor antagonists or sartans in treatment of chronic/diabetic associated kidney diseases. Blockade of AT₁ receptors directly causes vasodilation, reduces secretion of vasopressin, reduces production and secretion of aldosterone, amongst other actions—the combined effect of which is reduction of blood pressure which contibution to progression of nephropathy in hypertensive patients with type 2 diabetes. Administration of rhGDF-5 at the same time as AT1 receptor antagonists would repair the damaged kidney tissue more efficiently within a corrected hemodynamic background in patients. Angiotensin receptor blocking drugs (ARBs) include losartan, irbesartan, and candesartan.

### EXAMPLE 4

One could further combines rhGDF-5 with antagonists for aldosterone receptor in treatment of kidney diseases. In addition to increasing evidence of the importance of aldosterone as a key target hormone in hypertension, aldosterone receptor antagonism has been shown to blunt the effect of activation of the renin-angiotensin-aldosterone system (RAAS) on nephrosclerosis, independent of effects on blood pressure. The aldosterone receptor antagonists include Spironolactone and Eplerenone.

### EXAMPLE 5

The efficacy of the combination therapy was tested in a diabetic nephropathy rat model.

Diabetic condition in the animals was induced for 1 week by a single i.v. injection of Streptozotocin. Subsequently, the animals were treated for 14 weeks with or without 300 μg/kg rhGDF-5 (i.p., 3× per week), 10 mg/kg of Enalapril (gavage, once daily), or a combination of the two.

Blood glucose measurements and subcutaneous insulin administrations (as necessary) were carried out thrice weekly to maintain a glucose level between 280 and 400 mg/dL. Urine samples were collected every two weeks; blood samples collected every four weeks for measuring level of urine microalbumin, serum creatinine, blood urea nitrogen, and urine creatinine. On week fourteen, a final set of samples was collected; then the animals were euthanized and subjected to a limited necropsy involving the collection of the kidneys. One kidney from each animal was flash frozen for gene expression analysis as needed and the other preserved in 10% neutral buffered formalin, after which it was examined microscopically.

Histological examination was performed on the kidneys. The fixed tissues were processed in paraffin, sectioned at approximately 4 microns and stained with hematoxylin and eosin (H&E), periodic acid-Schiff's stain (PAS) and Masson's trichrome stain. Slides were evaluated on the basis of morphologic changes according to the following grading scheme: 0=within normal limits; 1=minimal; 2=mild; 3=moderate. All microscopic slides were evaluated and gross and microscopic findings were reported.

While the data were not statistically significant on all parameters examined, there is a positive trend that combination of rhGDF-5 and Enalapril show marginal effects on increasing survival rate (FIG. 3A), reducing tubular epithelial cell vaculolation (FIG. 3B) and BUN level at early time point (FIG. 3C). rhGDF-5 alone has comparable effect on reducing protein level in urine (FIG. 3D).

### REFERENCES

1. Sweitzer, S. M., Peters, M. C., Ma, J. Y., Kerr, I., Mangadu, R., Chakravarty, S., Dugar, S., Medicherla, S., Protter, A. A., and Yeomans, D. C. 2004. Peripheral and central p38 MAPK mediates capsaicin-induced hyperalgesia. Pain 111 :278-285.
2. Sweitzer, S. M., Medicherla, S., Almirez, R., Dugar, S., Chakravarty, S., Shumilla, J. A., Yeomans, D. C., and Protter, A. A. 2004. Antinociceptive action of a p38alpha MAPK inhibitor, SD-282, in a diabetic neuropathy model. Pain 109:409-419.
3. Heuvel, J. P. V. Sep. 24, 1997. PCR Protocols in Molecular Toxicology (METHODS IN TOXICOLOGY) (Paperback): CRC.
4. Lloyd, R. V. Jun. 15, 2001. Morphology Methods: Cell and Molecular Biology Techniques (Hardcover): Humana Press.

We claim:

1. A method of treating conditions which place mammals at a risk of acute or chronic renal failure, the method comprising administering a combination of at least one morphogen, stem cells and an anti-inflammatory or an inhibitor of the renin-angiotensin-aldosterone system.

2. The method of claim 1 wherein said morphogen is rhGDF-5.

3. The method of claim 2 wherein said rhGDF-5 is administered intravenously, intraperitoneal or by renal artery or subcapsular injection, or locally by implantation in matrices.

4. The method of claim 1 wherein said stem cells are bone marrow mesenchymal.

5. The method of claim 1 wherein said stem cells are adult stem cells from any tissue.

6. The method of claim 1 wherein said stem cells are from any developmental stage.

7. The method of claim 1 wherein said stem cells are renal cells induced with morphogens.

8. The method of claim 1 wherein said inhibitors of RAAS is ACE inhibitors,

9. The method of claim 1 wherein said inhibitors of RAAS is rennin inhibitors.

10. The method of claim 1 wherein said inhibitors of RAAS is aldosterone receptor antagonists.

11. The method of claim 1 wherein said anti-inflammatories are from the 38 MAP kinase inhibitors.

12. The method of claim 1 wherein said anti-inflammatories are antibodies against pro-inflammatory cytokines.

13. The method of claim 1 wherein said anti-inflammatories are antibodies against chemoattractant molecules.

14. The method of claim 1 wherein said anti-inflammatories are cell cycle inhibitors.
